(19) United States
(12) Patent Application Publication
(10) Pub. No.: US 2008/0243112 A1
(43) Pub. Date: Oct. 2, 2008

(54) Device and Method For Assisting Heat Ablation Treatment of the Heart

(76) Inventor: Werner Francois De Neve, Aalst (BE)

Correspondence Address: KNOBBE MARTENS OLSON & BEAR LLP, 2040 MAIN STREET, FOURTEENTH FLOOR, IRVINE, CA, 92614, US

(21) Appl. No.: 12/064,225

(22) PCT Filed: Aug. 19, 2005
(86) PCT No.: PCT/EP05/09007

§ 371 Date: Feb. 19, 2008

(51) Int. Cl. A61F 7/12; A61B 18/04

(52) U.S. Cl. 606/28; 607/105

(57) ABSTRACT

A heat exchange balloon assembly (11) for cooling the esophagus during heat ablation treatment to the heart is disclosed. The heat exchange balloon assembly includes an inflatable balloon (4) adapted for insertion into the esophagus, provided with an exterior heat-transfer surface and a lumen within the inflatable balloon (4) adapted to carry thermal exchange medium configured such that said heat-transfer surface conducts thermal energy between the esophagus and the lumen. Also disclosed is a heat exchange balloon assembly (11) connected to a temperature controller provided with a means to receive data pertinent to the power supplied to an ablation probe and/or the temperature of the tip of the ablation probe. The device may be used in an ablation system and in a method for treatment of atrial fibrillation.

### BACKGROUND TO THE INVENTION

Atrial fibrillation (AF) is the most common sustained cardiac arrhythmia encountered in clinical practice. It affects almost 2.3 million individuals in the USA alone. In the last 15 years, hospital admissions resulting from AF have increased two to three fold. This increasing prevalence is more apparent among elderly patients and is higher in men than in women. AF is an independent predictor of mortality and it is associated with an increased incidence of embolic stroke. For these reasons, AF is considered to be one of the three growing epidemics in the 21st century.

Many approaches have been described in recent years for the treatment of AF by heat ablation e.g. delivered by ultrasound, a laser or by radiofrequency (RF). Most of them are intended to electrically isolate the pulmonary veins from the left atrium and to draw atrial and left isthmus lines by burning the atrial tissue with an ablation probe. The effectiveness of these approaches to control AF ranges between 70% and 85% in subjects with otherwise healthy hearts, without the need of using antiarrhythmic drugs. This treatment is more effective than antiarrhythmic drugs alone.

Complications of this procedure are those inherent to any cardiac catheterisation procedure, for example: bleeding, pericardial effusion, cardiac tamponade, neumothorax, hemothorax, etc. And those inherent to heat ablation on the left atrium, being these: puncture of the aorta during transeptale puncture, clot formation and systemic embolisation, pulmonary vein stenosis, etc. Another complication is the development of a communication between the left atrium and the esophagus (atrio-esofageal fistula) due to a burning lesion applied in the posterior wall of the left atrium that indirectly burns the anterior wall of the esophagus. In most of the reported cases, this complication was lethal. For this reason physicians are very concerned when applying heat ablation in the posterior wall of the left atrium. Sometimes for safety reasons they avoid delivering heat ablation in areas of the heart that are close to the esophagus in this way administering incomplete or sub-optimal therapy.

Methods have been developed in the art to minimise the chances of developing an atrio-esofageal fistula. Cardiac CT scans or MRI scans to locate the esophagus and determine its relation to the left atrium are being performed prior to the ablation procedure. However, because the location of the esophagus varies with swallowing and respiration, these imaging techniques are not useful in preventing this complication. Other approaches measure the esophageal temperature during the ablation procedure. If the temperature increases the treatment is halted. The rise in esophageal temperature has proven to be a late warning sign since many times the device measuring the temperature is far from the site were heat is being applied. Other therapies have used the expensive intracardiac echocardiogram to titrate the power given during ablation. When they observe micro-bubble formation (a sign of left atrial tissue overheating) they reduce the power or they stop the application. Unfortunately the sensitivity of this method is low. Only 40% of the ablation applications that increase esophageal temperature have shown to generate micro-bubble formation on intracardiac echo.

In view of the prior art, it is not possible to neither predict nor prevent the generation of atrio-esophageal fistulas with the present technology. This raises the need of developing new devices and techniques to safely carry out ablation of atrial fibrillation without developing an atrioesophageal fistula, which the present invention provides.

### BRIEF DESCRIPTION OF THE FIGURES

FIG. 1: A longitudinal cross section of a heat exchange balloon assembly according to the present invention.

FIG. 2: A longitudinal cross section of an alternative heat exchange balloon assembly, provided with a fluid exit port.

FIG. 3: A three dimensional representation of an alternative heat exchange balloon assembly, provided with a fluid exit port and a tubing coupling.

FIG. 4: A three dimensional drawing showing the relative position of the heart and oesophagus, and an example of an optimum position of a heat exchange balloon assembly.

FIG. 5: A schematic drawing of a heat exchange balloon assembly, temperature controller, data receiving means and ablation device.

### SUMMARY OF THE INVENTION

One embodiment of the present invention is a heat exchange balloon assembly (11) for cooling the oesophagus during heat ablation treatment to the heart comprising:
an inflatable balloon (4) adapted for insertion into the oesophagus, provided with an exterior heat-transfer surface; and
a lumen within said inflatable balloon (4) adapted to carry thermal exchange medium;
   configured such that said heat-transfer surface conducts thermal energy between oesophagus and said lumen.

During operations where heat ablation to the heart is performed, the use of a heat exchange balloon assembly placed the oesophagus prevents or limits damage to the oesophagus caused by heat. This allows the skilled practitioner to perform ablation treatment to completion, without undue concern for damage to the oesophagus. Another embodiment of the present invention is a balloon assembly as described above, comprising:

(a) a first elongate tubular body (1) having a proximal end (9) and a distal end (10);
(b) a second elongate tubular body (2) having a proximal end (9) and a distal end (10);
wherein said inflatable balloon (4) is in fluid communication with the distal ends of the first elongate tubular body (1) and the second elongate tubular body (2).

Another embodiment of the present invention is a balloon assembly as described above, comprising:

(a) an elongate tubular body (1) having a proximal end (9) and a distal end (10);
(b) a fluid exit port (22);
wherein said inflatable balloon (4) in fluid communication with the distal end (10) of the elongate tubular body (1) and said fluid exit port (22) is in fluid communication with a distal end (10) of the balloon (4).

In normal use, the thermal exchange balloon assembly is connected to a temperature controller for pumping thermal exchange medium to said assembly, and receipt thermal exchange medium returning from said assembly. The use of a fluid exit port simplifies the design of tubing, in that a second elongate tubular body is unnecessary. Furthermore, a greater area of cooling is achieved by the contact of cooled waste thermal exchange medium with the oesophageal wall.

Another embodiment of the present invention is a balloon assembly as described above wherein the fluid exit port is provided with a means (38) to prevent thermal exchange medium draining into the oesophagus until after the balloon (4) has inflated.

Another embodiment of the present invention is a balloon assembly as described above wherein the balloon (4) comprises an outer lumen (34) configured to carry thermal exchange medium, and a hollow inner lumen (35).

Another embodiment of the present invention is a balloon assembly as described above wherein, wherein at least one elongate tubular body terminates in a tubing coupling (31).

Another embodiment of the present invention is a balloon assembly as described above wherein, wherein at least one elongate tubular body is configured to connect to a temperature controller (51).

Another embodiment of the present invention is a balloon assembly as described above, connected to a temperature controller (51) provided with a means to receive data (52) from an ablation device (54).

Another embodiment of the present invention is a balloon assembly as described above, wherein said data comprises an indication of power supplied to an ablation probe (55) of the ablation device (54) and/or an indication of the temperature of the ablation probe (55).

Another embodiment of the present invention is a balloon assembly as described above, wherein the data receiving means (52) comprises a processor configured to process said data, and output a signal to the temperature controller (51) useful for the adjustment of the temperature of the heat exchange medium.

Another embodiment of the present invention is a balloon assembly as described above, wherein said temperature controller (51) and means to receive data (52) are configured to adjust the temperature of the heat exchange medium according to the power supplied to the ablation probe (55) and/or the temperature of the ablation probe (55). The temperature controller reacts to power used during ablation and to the temperature of the probe in order to modulate the temperature of the oesophagus before damage occurs thereto.

Another embodiment of the present invention is a balloon assembly as described above, wherein the temperature of heat exchange medium is adjusted to maintain a time-averaged temperature of the oesophagus during ablation of less than 37 deg C. By compensating fluctuations in the temperature caused by local heating with an increased cooling of the balloon, the time averaged average temperature of the oesophagus is maintained so as to prevent heat damage.

Another embodiment of the present invention is a balloon assembly as described above, wherein the time-averaged temperature of the oesophagus during ablation is between 25 to 34 deg C.

Another embodiment of the present invention is a temperature controller (51) suitable for use with a heat exchange balloon assembly as described above, comprising means to adjust the temperature of the heat exchange medium and a means to receive data (52) from the ablation device (54).

Another embodiment of the present invention is a temperature controller (51) as described above configured to adjust the temperature of the heat exchange medium according to the power output an ablation device (54) and/or the temperature of the ablation probe (55).

Another embodiment of the present invention is a temperature controller (51) as described above, wherein the temperature of heat exchange medium is adjusted to maintain a time-averaged temperature of the oesophagus during ablation of less than 37 deg C.

Another embodiment of the present invention is a temperature controller (51) as described above, wherein the time-averaged temperature of the oesophagus during ablation is between 25 to 34 deg C.

Another embodiment of the present invention is an ablation system comprising a heat exchange balloon assembly (11) as described above, a temperature controller (51), a means to receive data (52) and an ablation device (54).

Another embodiment of the present invention is an ablation system as described above, configured to adjust the temperature of the heat exchange medium according to the power output of the ablation probe (55) and/or the temperature of the ablation probe (55).

Another embodiment of the present invention is an ablation system as described above, wherein the temperature of heat exchange medium is adjusted to maintain a time-averaged temperature of the oesophagus during ablation of less than 37 deg C.

Another embodiment of the present invention is an ablation system as described above, wherein the time-averaged temperature of the oesophagus during ablation is between 25 to 34 deg C.

Another embodiment of the present invention is a use of a heat exchange balloon assembly (11) as described above for cooling the oesophagus during heat ablation treatment.

Another embodiment of the present invention is a method for the safe treatment of atrial fibrillation by heart ablation using a heat ablation device (54) comprising the steps of:
1) Inserting a heat exchange balloon assembly (11) as described above, into the oesophagus of a subject,
2) adjusting the temperature of the heat exchange medium according to the power supplied to an ablation probe (55) of said ablation device (54), and/or according to the temperature of the ablation probe (55), so as to lower the temperature of the oesophagus during heat ablation treatment to the heart.

Another embodiment of the present invention is a method as described above, wherein the temperature of the oesophagus is maintained at a time-averaged temperature of less than or equal to 37 deg C.

Another embodiment of the present invention is a method as described above, wherein the time-averaged temperature of the oesophagus is between 25 and 34 deg C.

Another embodiment of the present invention is a method as described above, wherein the temperature of the heat exchange medium is further adjusted according to the reading of a temperature sensor located in or on the balloon (4).

Another embodiment of the present invention is a method as described above, wherein the temperature of the heat exchange medium is further adjusted according to the desire of the physician.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one, the grammatical object of the article. By way of example, “a valve” means one valve or more than one valve.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of samples, and can also include 1.5, 2, 2.75 and 3.80, when referring to, for example, concentrations).

The present invention relates to a method and device to assist carrying out heat ablation of atrial fibrillation, which cools the anterior wall of the oesophagus during ablation. The inventors have found cooling the anterior wall of the oesophagus prevents damage to the wall of the oesophagus during heat ablation. Heat ablation can thus be performed without undue concern for developing an atrioesophageal fistula.

Accordingly, a first aspect of the present invention is a heat exchange balloon assembly suitable for insertion into the oesophagus comprising an inflatable balloon provided with an exterior heat-transfer surface and a lumen adapted to carry thermal exchange medium, for cooling the oesophagus during heat ablation of the heart using an ablation probe. According to one aspect of the invention, said heat-transfer surface conducts thermal energy between oesophagus and the lumen. Preferably, the lumen is formed at least in part by an interior surface of said heat transfer surface. The heat exchange balloon removes heat from the wall tissue of the oesophagus, and thereby cooling the oesophagus, so precluding damage during heat ablation of the heart.

The first aspect of the invention provides a heat exchange balloon adapted for placement within oesophagus of a mammalian subject, wherein the heat exchange balloon effects in situ heat exchange between the heat exchange balloon and the oesophagus, thereby altering and/or maintaining a low temperature of at least part of the oesophagus and/or region in contact with the oesophagus. The heat exchange balloon can be any suitable balloon adapted for insertion in to the oesophagus and comprising a heat exchange surface. Such balloons are described in the art, such as, for example, as disclosed in US 2004/210281, U.S. Pat. No. 6,755,849, US 2004/0210278, U.S. Pat. No. 6,604,004, U.S. Pat. No. 5,716,386, U.S. Pat. No. 5,496,271 which are incorporated herein by reference.

The heat exchange balloon generally comprises an inflatable balloon, which surface when inflated contacts the wall of the oesophagus. Thermal exchange medium is provided to at least the surface of the balloon by means of supply tubing. According to one aspect of the invention, thermal exchange medium exits the balloon via exit tubing which is adjacent to the supply tubing. The supply and exit tubing may be bundled in effectively a single tubing member, or can be separate individual tubes. The tubing and balloon may be configured to enter the oesophagus through the mouth or nose of a patient. According to one aspect of the invention, the balloon is provided with a tubing coupling, which connects the balloon to the supply and optionally the exit tubing. Such coupling allows the balloon to reversibly disconnect from the tubing. According to another embodiment of the invention, the balloon and tubing are a single unit.

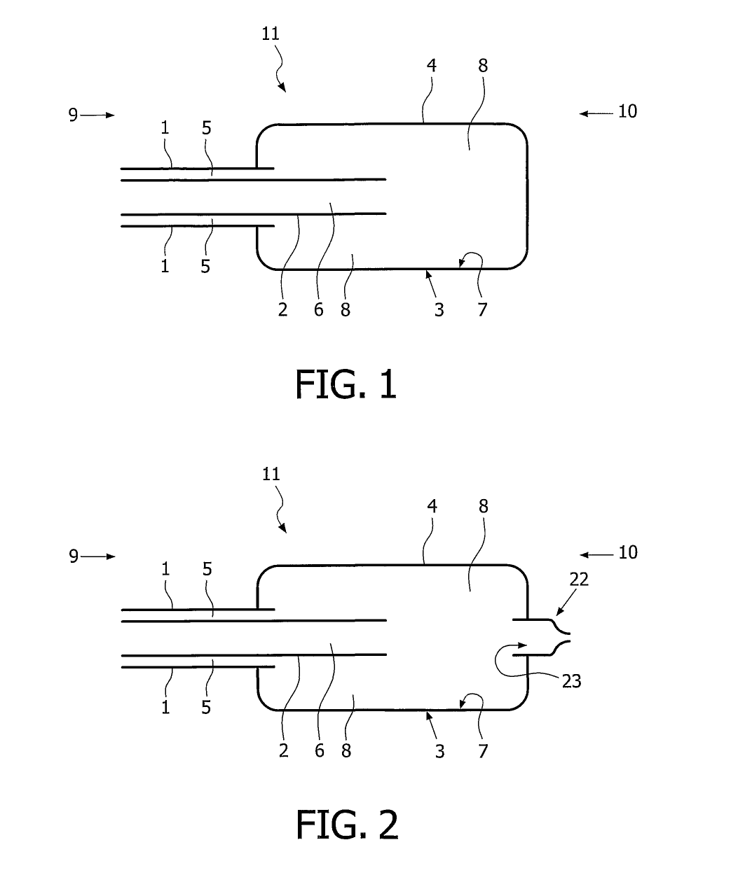

By means of a non-limiting example, a heat exchange balloon assembly 11 according one aspect of the present invention is shown in FIG. 1, which depicts a longitudinal cross-section of a balloon assembly. A heat exchange balloon assembly 11 comprises:

(a) a first elongate tubular body 1 having a proximal end 9 and a distal end 10;
(b) a second elongate tubular body 2 having a proximal end 9 and a distal end 10; and (c) a balloon 4 in fluid communication with the distal ends of the first elongate tubular body 1 and the second elongate tubular body 2. In FIG. 1, the second elongate tubular body 2 is disposed longitudinally within the first elongate tubular body 1. As mentioned elsewhere, these first and second elongate tubular bodies can be arranged in any suitable manner, for example, as separate tubes or as a single tubular bundle. The balloon 4 is affixed to the outer surface of the first elongate tubular body in proximity to the distal end. The balloon defines an inflation lumen 8 that is fluidly connected to the lumens 5, 6 of the first elongate tubular body 1 and second tubular elongate body 2. The balloon has an outer surface 3 and an inner surface 7 and is adapted to conform in shape to the oesophagus, such that when inflated, the outer surface 3 of the balloon is in contact with a surface of the oesophagus and forms a heat exchange surface with the surface of the oesophagus. The inner surface 7 of the balloon forms a heat exchange surface with a thermal exchange medium within the balloon 4. The material of the balloon 4 conducts heat such that heat is conducted from one heat exchange surface to the other.

Prior to ablation, the heat exchange balloon assembly 11 is inserted into the oesophagus. A thermal exchange medium is pumped through one of the elongate tubular bodies (e.g. 2) into the balloon 4. The balloon 4 expands, filling the lumen of the oesophagus and cooling the surrounding tissue. The thermal exchange medium exits the balloon via another of the elongate tubular bodies (e.g. 1) and in some embodiments, may be cooled and re-circulated. The thermal exchange medium may be a solid composition, a gel, a liquid, and a gas suitable for transferring heat energy. Changing the temperature of the thermal exchange medium or altering its flow rate alters the temperature of the target region. A pump may be employed to circulate the fluid in the tubular bodies, and the fluid flow rate can be regulated by adjusting the pumping rate, in this way modifying the temperature inside the balloon.

In one embodiment the first and second elongate tubular bodies may not be concentric, but may be separate and independent. In some embodiments the tubular bodies may be of different cross-sectional areas. In another embodiment, the first and/or second elongate tubular bodies may be shortened and terminate in a tubing coupling. The coupling allows the balloon 4 to be essentially disconnected from the first and/or second elongate tubular bodies that pass through the mouth or nose. In other embodiment, the heat exchange balloon assembly may possess one or more additional elongate tubular bodies which pass though the distal 10 wall of the balloon 4 and open out into the oesophagus. The heat exchange balloon assembly may additionally include a transducer (which may also be called a sensor or probe) for example an ultrasound visualising transducer. A transducer may be any device that measures a physical or physiological parameter such as temperature to monitor the effectiveness of the cooling process, pressure, electromagnetic fluctuations or sound that may be used in clinical monitoring of cardiac function. The transducer may be affixed, for example, to the distal end of a third elongated tubular body. The transducer may monitor the internal temperature of the balloon. The transducer may be used to locate the position of the balloon inside the oesophagus in relation to the heart. The heat exchange balloon assembly may additionally include a guide wire disposed longitudinally within a third elongate tubular body, the guide wire having a proximal end and a distal end. Additionally, a guide sheath may be fitted over at least a portion of the first elongate tubular body, the guide sheath having a proximal end and a distal end. In some embodiments, the present invention may include a digestible composition affixed to the distal end of the guide-wire to facilitate placement of the guide-wire in the oesophagus. The digestible composition attached to the guide-wire is placed in the mouth of the subject, the subject swallows the digestible composition, thereby bringing the guide-wire into placement in the oesophagus.

A variation of a heat exchange balloon assembly is an embodiment wherein an elongate tubular body supplies thermal exchange medium to the balloon, and thermal exchange medium exits the balloon through an opening in distal end of the balloon, flowing into the oesophagus and stomach. The arrangement requires only a single elongate tubular body for the supply of thermal exchange medium, which simplifies the design and is cost effective. Furthermore, the single tube design facilitates insertion through the mouth or nose by virtue of a thinner and more flexible elongate tubular body.

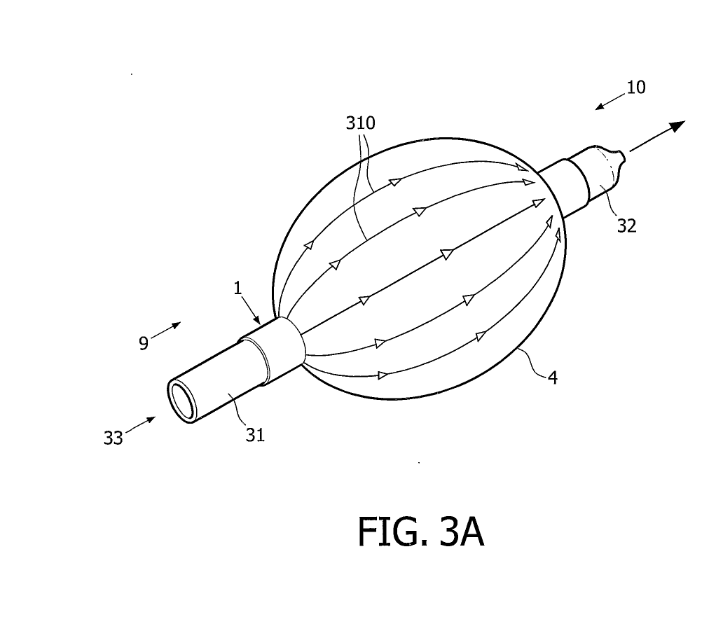

By means of a non-limiting example, a heat exchange balloon assembly 11 comprising a single elongate tubular body according to the embodiment shown in FIG. 2, which is a similar to the device shown in FIG. 1 except in the following features. The heat exchange balloon assembly 11 comprises (a) an elongate tubular body 1 having a proximal end 9 and a distal end 10; (b) a balloon 4 in fluid communication with the elongate tubular body 1 and (c) a fluid exit port 22 in fluid communication with the distal end 10 of the balloon. The proximal end of the balloon 4 is affixed to the outer surface of the elongate tubular body. The distal end of the balloon 4 is affixed to the outer surface of the fluid exit port 22. A lumen 8 is fluidly connected to the lumen 5, of the first elongate tubular body 1 and lumen 23 of the fluid exit port 22. After inflation by the thermal exchange medium, the outer surface 3 of the balloon is in contact with a surface of the oesophagus, and excess thermal exchange drains from the fluid exit port 22. The balloon may be provided with a system, such as a valve which prevents thermal exchange medium draining into the oesophagus until after the balloon 4 has inflated. Said system may be incorporated, for example, within the fluid exit port 22.

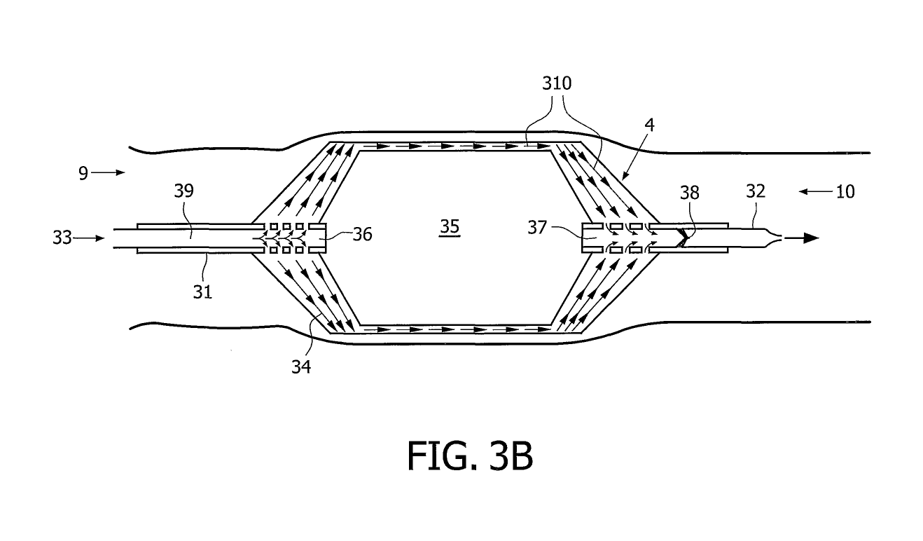

Another variation of the invention is where the balloon comprises an outer lumen configured to carry thermal exchange medium, and a hollow inner lumen. The hollow inner lumen may be essentially air filled. It can be inflated by air, or can passively fill with air during inflation of the outer lumen. The hollow inner lumen reduces the volume of thermal exchange medium inflating the balloon so reducing the weight of the oesophagus on the heart during ablation. Furthermore, the mixing and diffusion of the warmed thermal exchange medium with cooler incoming medium in the smaller volume of the outer lumen is more efficient. Furthermore, incoming, cooled thermal exchange medium is in closer contact with the inner wall of the balloon. A balloon according to this aspect of the invention is illustrated in FIGS. 3A and 3B. The balloon 4 in FIG. 3A is provided at the proximal end 9 with a single elongate tubular body 1 which terminates in a tubing coupling 31. The tubing coupling is suitable for connection to a reciprocating coupling of a single elongate tubular body which passes out of the subject, e.g. through the mouth or nose. Alternatively, the balloon and tubing may be a single unit. At the distal end 10 of the balloon 4, a fluid exit port 32 is provided. Thermal exchange medium enters through an opening 33 in the coupling 31, and fills the outer lumen 34 (FIG. 3B) of the balloon 4, and flows in the direction 310 of the distal end 10 of the balloon 4. Thermal exchange medium exits the outer lumen 34 via the fluid exit port 32. Entry of medium into the outer lumen 34 causes the balloon to inflate, and creates an air-filled void in the inner lumen. The inner lumen preferably comprises an air vent towards the proximal 9 end of the balloon. FIG. 3B shows a longitudinal cross section of the balloon of FIG. 3A, which clearly indicates the hollow inner lumen 35. The lumen 39 of the tubing coupling 31 is in fluid communication with the outer lumen 34 of the balloon 4; the lumen 37 of the fluid exit port 32 is in fluid communication with the outer lumen 34 of the balloon 4. The fluid exit port 32 is disposed with a means (e.g. valve) 38 to restrict the drainage of excess thermal exchange medium into the oesophagus until after the balloon 4 has inflated.

Variations of the balloon assembly 11 such as division of lumens in the balloon 4, the presence of ducts and openings to direct the internal flow of thermal exchange medium in the balloon 4, the presence of air inflation lumens and supply tubing, presence of a safety valve, air venting valve etc. can be readily incorporated by the person skilled in the art, and are within the scope of the present invention.

Depending on the configuration of the balloon assembly, the thermal exchange medium may be a gas, such as, but not limited to, gases used in refrigerant arts, for example, nitrous oxide (Cryo-Chem, Brunswick, Ga.), Freon™, carbon dioxide, nitrogen, and the like. The thermal exchange medium can be a liquid such as saline solution. The thermal exchange medium can be a gel, such as a gel that has a high specific heat capacity. Such gels are well known to those of skill in the art (see, for example, U.S. Pat. No. 6,690,578). Alternatively, a slurry may be used such as a mixture of ice and salt. The thermal exchange medium can be a solid, such as ice or a heat conducting metal such as, but is not limited to, aluminium or copper. An additional embodiment of the invention envisions a combination of different thermal exchange media, such as, but is not limited to, a liquid-solid heat exchange combination of saline solution and aluminium metal shaped into fins.

In yet another embodiment of the invention, the thermal exchange medium comprises two or more chemical mediums separately located in the catheter lumens that, when mixed, remove heat from the environment. Examples of such two chemical media are ammonium nitrate and water, but are not limited to these mediums. When ammonium nitrate and water are mixed an endothermic reaction occurs and heat is taken up by the reagents in a predictable manner. In yet another embodiment of the invention, the thermal exchange medium comprises two chemical compositions separately located in the catheter lumens that, when mixed, generate heat. Examples of such two chemical media are magnesium metal and water, but are not limited to these mediums. When magnesium metal and water are mixed an exothermic reaction occurs and heat is released in a predictable manner. Additional chemical mediums that improve the rate of reaction are known to those of skill in the art.

The elongate tubular bodies may be constructed of any suitable materials sufficiently flexible so as to be able to follow and conform to the natural shape of the oesophagus, but sufficiently stiff to hold its generally linear shape while being pushed into the oesophagus.

The balloon can be constructed of materials sufficiently flexible so as to be able to follow and conform to the natural shape of the oesophagus, such as latex rubber, elastic, or plastic.

According to one embodiment of the invention, the balloon assembly may comprise at least one imaging marker such as a radio-opaque substance situated at a known position on or within the assembly, for example at either end of the balloon. These markers can be used to view the position of the balloon when inserted into a subject. The markers include, but are not limited to radio-opaque compounds, fluorescent compounds, radioactive compounds or similar compounds.

According to one aspect of the invention, oesophagus may be cooled at a rate of between about 0.5 deg C./hour and 30 deg C./hour, or about 1.0 deg C./hour and 20 deg C./hour, or about 2.0 deg C./hour and 10 deg C./hour, preferably at a rate of about 3 deg C./hour to about 5 deg C./hour. The target organ also may be cooled by at a rate of between about 0.5 deg C./30 minutes and 30 deg C./30 minutes, or about 1.0 deg C./30 minutes and 20 deg C./30 minutes, or about 2.0 deg C./30 minutes and 10 deg C./30 minutes, preferably at a rate of about 2 deg C./30 minutes to about 5 deg C./30 minutes.

The temperature of the thermal exchange medium is changed to anticipate the increase in temperature of the oesophagus caused by heart ablation. Therefore, the invention adjusts the temperature and/or flow rate of the heat exchange medium, affecting the temperature of the balloon 4 so that ultimately the temperature of the oesophagus remains between 25 to 34 deg C. Before local heating of the oesophagus arises due to ablation, the temperature of the balloon is lowered so that the oesophagus is further cooled before a rise in temperature occurs. Alternatively, the temperature of oesophagus may be maintained at a constant low temperature by meeting the rise in temperature of the oesophagus simultaneously with a decrease in temperature of the balloon.

According to another aspect of the invention, the temperature of the thermal exchange medium is adjusted so as to maintain an essentially constant average temperature of the oesophagus during ablation. The time over which the temperature is averaged may be less than or equal to 0.5 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 40 s, 50 s, 60 s, 70 s, 80 s, 90 s, 100 s, 110 s, 120 s, 150 s, 180 s, 210 s, 240 s, 5 min, 6 min, 7 min, 8 min, 9 min, 10 min, or at an interval between any two of the aforementioned times. The time interval over which the average temperature is taken is preferably between 0.5 s and 240 s.

The time-averaged temperature of the oesophagus in the region of the balloon may be less than or equal to 2, 3, 4, 5, 6, 7, 8, 9, 10, 11, 12, 13, 14, 15, 16, 17, 18, 19, 20, 21, 22, 23, 22, 25, 26, 27, 28, 29, 30, 31, 32, 33, 34, 35, 36, 37 or 38 deg C., or at a temperature between any two of the aforementioned temperatures. The time-averaged temperature of the oesophagus in the region of the balloon is preferably between 25 and 34 deg C.

According to another aspect of the invention, the temperature of the thermal exchange medium may be adjusted so as to prevent the temperature of the oesophagus during ablation exceeding a maximum temperature. The maximum temperature may be about 10, 11, 12, 13, 14, 15, 16, 17, 18, 19, 20, 21, 22, 23, 22, 25, 26, 27, 28, 29, 30, 31, 32, 33, 34, 35, 36, 37, 38, 39, or 40 deg C., or at a temperature between any two of the aforementioned temperatures. The maximum temperature of the oesophagus in the region of the balloon which should not be exceeded is preferably between 36 and 40 deg C.

According to an aspect of the invention, the temperature and flow rate at which the heat exchange medium passes through the balloon to achieve a temperature of the oesophagus can be calculated by the person skilled in the art, taking into account factors such as balloon size, balloon material, tubing length and material, ablation power and temperature of the ablation probe (i.e. the tip of the probe).

According to one aspect of the invention, the balloon 4 is maintained at a temperature between 25 to 34 deg C. when the supply of power to the probe 55 of less than 30 watts. When a power greater than or equal to 30 watts is supplied to the probe 55, the temperature and/or flow rate of thermal exchange medium is changed so as to reduce the temperature of the balloon by 0.5 to 7 deg C., but preferably by 0.5 to 2 deg C. per additional 5 watts increase in the power.

Where the ablation probe 55 is fitted with a temperature sensor that monitors the temperature of the probe (i.e. the tip of the probe), the temperature of the balloon 4 may be adjusted for any increase in the temperature registered by the ablation probe. Above 50 degrees C., any increase in the temperature registered by the ablation probe may be counteracted by a programmable increase in the flow rate and/or temperature of the thermal exchange medium, in order to reduce the temperature of the balloon between 0.5 degrees to 7 degrees, but preferably by 0.5 to 2 degrees, per 2 degrees C. increase in the temperature registered by the ablation probe.

Where in the balloon 4 is provided with an interior and/or exterior temperature sensor, the flow rate and/or temperature of the thermal exchange medium may be changed in order to achieve the desired balloon temperature. Monitoring the balloon temperature allows more directly losses due, for example, to the tubing 1 length, tubing 1 insulation, and room temperature to be corrected. The temperature of the balloon may also be adjusted according to the treating physician's desire.

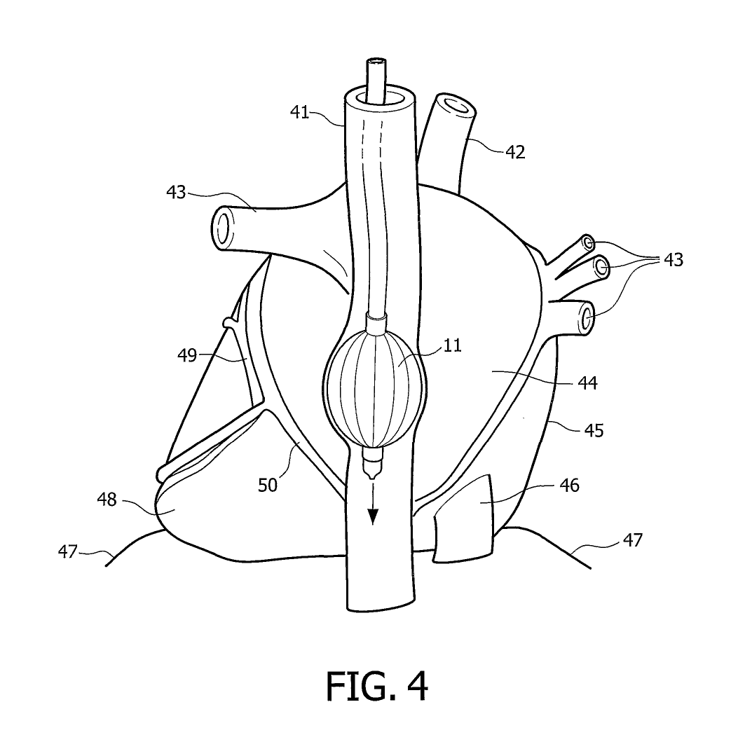

The oesophagus is anatomically positioned adjacent to the heart. When heat ablation is performed on the heart, heating of the anterior wall of the oesophagus is prevented. The precise position of the balloon will depend on the area of the heart undergoing heat ablation. FIG. 4 shows the relative position of the heart and oesophagus within the thoracic cavity in a posterior view, and an example of an optimum position of a heat exchange balloon assembly 11 for performing heat ablation of the left atrium 44. The oesophagus 41 is positioned adjacent to the myocardium of the left atrium 44. Also shown are the superior vena cava 46, the pulmonary veins 43, the inferior vena cava 46, the diaphragm 47, the left coronary artery 49 and the left coronary vein 50, the left atrium 44 and right atrium 45.

Regulating the temperature of the oesophagus during ablation of the heart is an entirely novel and inventive concept, and in its simplest embodiment the invention encompasses a device for cooling the oesophageal during heart ablation of a subject comprising: a reservoir adapted in shape and size to conform to the lumen of the oesophagus, and a thermal exchange medium disposed within the reservoir. The device may optionally include one or more tubes in fluid communication with the balloon. These tubes may be used to transmit the thermal exchange medium into and out of the balloon. The thermal exchange medium may be pumped through the tubes and the balloon using a conventional pump, generally present at the proximal end of the tubes, outside the patient being treated. Alternatively, the thermal exchange medium may not be circulated, but may be contained statically within the balloon.

A second aspect of the present invention is a heat exchange balloon assembly 11 as described herein, connected to a temperature controller provided with a means to receive data from an ablation device. As the power supplied to an ablation probe can vary during heat ablation to the heart, so the temperature of the thermal exchange medium and/or its flow rate is adjusted to change the temperature of the balloon. The temperature and/or flow rate of the thermal exchange medium can be adjusted according to the power supplied to the ablation probe and/or the temperature at the tip of the ablation probe and/or according to the treating physicians expert knowledge. The temperature inside the balloon may be monitored so that losses during the passage of the heat exchange medium to the balloon can be corrected. Thus, the assembly is configured to adjust and maintain the temperature of the balloon 4 according to the power supplied to the ablation probe or to the other parameters above mentioned. By compensating for an increased heating effect of the oesophagus by the ablation probe before the oesophagus tissue becomes heated, heat damage to the oesophagus is circumvented. Treatments of the prior art rely on detecting a change in temperature at the oesophagus, however, by the time a temperature limit is exceeded, damage to the tissue is already made. The present invention overcomes this by using information regarding the power supplied to the probe, and/or the temperature of the probe.

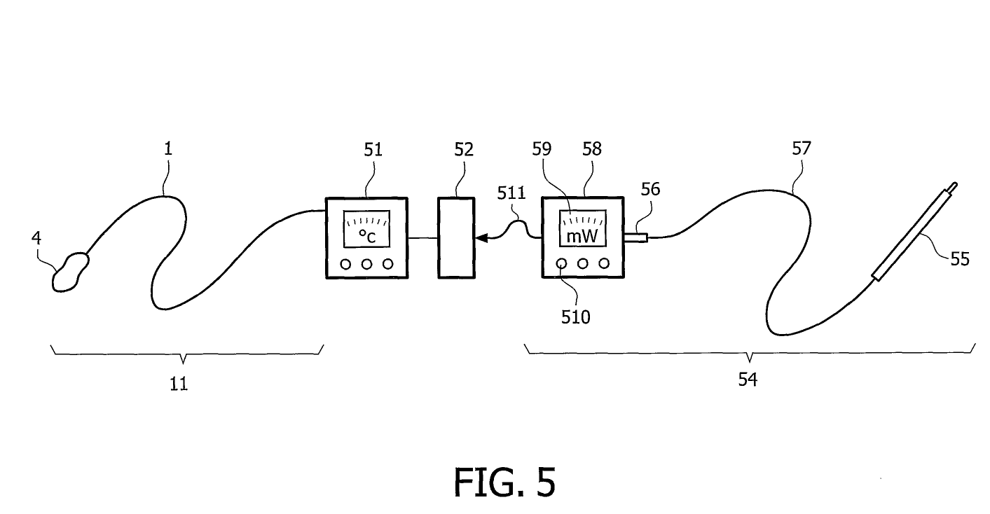

By means of a non-limiting example, FIG. 5 depicts schematic illustration of a heat exchange balloon assembly 11 comprising a temperature regular 51 and a means 52 to receive data 53 from an ablation device 54.

### Data

The data 511 may comprise an indication of the power supplied to the ablation probe 55 of the ablation device 54. The data 511 can also be indication of the power output to the probe 55 of the ablation device 54. It can be, for example, an electronic signal such as a digital or analogue signal. An inline coupling to the ablation probe 55, may provide data in the form of an amplitude signal proportionate to the power supplied to the ablation probe 55. The data 511 can be information read from a power setting dial 510 or display 59 of the ablation device 54. The data 511 might be readily available from the ablation device 55, for example, through a serial or parallel computer port. Data may also be readings from a temperature sensor fitted to the ablation probe 55.

### Means to Receive Data

The means to receive data 52 can be any means for receiving an indication of power supplied to the probe 55 and/or the temperature of the probe. The means can comprise, for example, a connection to a port on the ablation device 54. The connection can be, for example, a serial or parallel computer interface port. It can be a connection to the power output 56 to ablation probe 55 itself. In the latter case, the means 52 may comprise additional electronic circuitry to convert the energy supply to the ablation probe into a power measurement e.g. via an analogue to digital converter (ADC). The data receiving means 52 can be an inputting means, such as a keyboard, for entering ablation probe power data, read from a dial 510 or display 59 on a controller 58 of the ablation device 54. Variation of the means to receive data 52 will depend on the specification of the ablation device 54, and can be readily determined by the person skilled in the art.

The means to receive data 52 can be incorporated at least partly into the temperature controller 51. Alternatively, it can be separate from the temperature controller 51, connecting therewith via one or more cables and/or connectors. Where it is separate, the means to receive data 52 preferably comprises a connector suitable for mating with the temperature controller 51, through which data and/or control signals pass. Similarly, the means to receive data 52 can be incorporated at least partly into the ablation device 54. Alternatively, it can be separate from ablation device 54; it can connect therewith via one or more cables and/or connectors where appropriate.

According to an aspect of the invention, the data receiving means 52 may comprise a processor configured to process data 511 and output a signal to the temperature controller 51, useful for the adjustment and constancy of the temperature of the balloon 4. The signal may be, for example, power data, temperature data and/or instructions to adjust and/or maintain the temperature and/or flow. For example, data 511 which indicates an increase in power supplied to the ablation probe 55, can be processed by the microprocessor which in turn produces a signal to decrease the temperature of the thermal exchange medium. Conversely, data indicating a decrease in power supplied to the ablation probe 55, can provide a signal to increase the temperature of the thermal exchange medium. Thus, according to an aspect of the invention, the means to receive data 52 comprises means for sending signals to the temperature controller 51 to change the temperature of the thermal exchange medium in response to the data 511 regarding ablation power and/or temperature of the ablation probe 55.

The data receiving means 52 preferably comprises the processor and other electronics such as an ADC, and interfaces with the temperature controller. Where the data receiving means 52 is connected to a temperature controller 51 already equipped with a processing means, some or all of the tasks of the data receiving means can performed by this processor. The temperature controller 51 and data receiving means 52 can be a single entity (e.g. a stand alone device) or a plurality of separate components (e.g. reservoir, cooling means, PC computer, electronic interface, connection to ablation device).

### Temperature Controller

The temperature controller 51 comprises means to adjust and maintain the temperature and/or flow rate of the thermal exchange medium supplied to the heat exchange balloon 4. Temperature controllers are well known in the art. Generally a temperature controller comprises a reservoir of thermal exchange medium, a cooling system such as a peltier device or cooling device based on gaseous refrigerant, and a regulating means. A pump or gravity is used to supply thermal exchange medium cooled by the cooling system to the balloon. The temperature controller 51 is capable of adjusting and maintaining the temperature of the thermal exchange medium. The flow rate of the thermal exchange medium may also be varied by the controller. The temperature controller may also be configured to maintain or adjust the temperature of the balloon 4 according to feedback received from a temperature sensor in or on the balloon 4. The temperature controller 51 can be equipped with a means for control by another device (e.g. by the data receiving means). It may comprise a processor and electronics which perform the task of the data receiving means, and can change and maintain the temperature of the balloon 4 according to the power supplied to the ablation probe 55.

### Ablation Device

A heat ablation generator, known herein as an ablation device, is well known in the art, and typically comprises a control unit 54 connected via cable 57 to an ablation probe 55. It can be provided with a plurality of controls 510 and dials 59 for adjusting at least the power output of the ablation probe 55. The control unit 54 comprises means to supply and control energy, to the ablation probe 55 such an amplifier. The ablation probe 55 may be provided with a temperature sensor at the tip for monitoring the actual temperature of the probe during ablation. The ablation probe is capable of delivering energy to the tissues of a subject, to form a burn therein. The energy most commonly used is radiofrequency energy, though other suitable energies include laser and infrared. The ablation probe can be a radiofrequency electrode, a visible light laser, an infrared laser, or any suitable probe for delivering controlled heat. Examples of ablation devices can be found, for example, in WO 97/32525 and WO 90/04709 which are incorporated herein by reference.

A third aspect of the present invention is a temperature controller as described above, further comprising means to receive data 52 from an ablation device 54. According one aspect of the invention, a temperature controller 51 suitable for use with an inflatable balloon assembly 11, comprises means to adjust and maintain the temperature of the thermal exchange medium, and a means to receive data from the ablation device 54.

The temperature controller may be configured to adjust the temperature of the thermal exchange medium according to the power output an ablation device 54.

The temperature controller may configured to adjust the temperature of the thermal exchange medium according to the temperature detected by a temperature sensor in the ablation probe of an ablation device 54.

The temperature controller may also be configured to adjust the temperature of the thermal exchange medium according to feedback received from a temperature sensor in or on the balloon 4. Such feedback allows compensation for heat losses in the tubing 1 where the tubing 1 is not insulated, or is long in length, or the operating environment is warmer or cooler compared with the heat exchange medium.

The controller may also be configured to adjust the temperature of the thermal exchange medium according to a combination of two or more of the aforementioned parameters. The balloon assembly 11, data 511, temperature controller 51 and means to receive data 52, and ablation device 54 are described above. Ways to connect and configure the above mentioned devices are known to the skilled person. The temperature controller 51 and means to receive data 52, can be a single entity (e.g. a stand-alone device) or a plurality of devices (e.g. a separate reservoir, cooling means, controller, and means to receive data). This third aspect of the invention, can be incorporated into other devices, for example, as part of the ablation device 54 or system.

A fourth aspect of the present invention is an ablation system comprising a heat exchange balloon assembly 11 as described herein, a temperature controller 51, means to receive data 52, and an ablation device 54. Preferably, said system is configured to adjust and maintain the temperature of the heat exchange medium according to the power output of the ablation probe 55. The system may be configured to adjust and maintain the temperature of the heat exchange medium according to the temperature detected by a temperature sensor in the ablation probe of an ablation device 54. The temperature controller may also be configured to adjust the temperature of the thermal exchange medium according to feedback received from a temperature sensor in or on the balloon 4. The system may be configured to adjust and maintain the temperature of the heat exchange medium according to the expert opinion of the treating physician. The system may also be configured to adjust and maintain the temperature of the heat exchange medium according to a combination of two or more of the aforementioned parameters. The heat exchange balloon assembly 11, data, temperature controller 51, means to receive data 52, and ablation device 54 are described above. The various devices can be incorporated into a single entity which comprises the above components. Alternatively, the ablation system can be a plurality of devices (e.g. a separate reservoir, cooling means, controller, means to receive data and ablation device). Way to connect and configure the above mentioned devices are known to the skilled person.

A fifth aspect of the present invention is a use of a balloon as described herein for cooling and/or maintaining the temperature of the oesophagus during heat ablation treatment. One aspect of the invention of a balloon as described herein for assisting heat ablation treatment of the heart. According to another aspect of the present invention, a method for the safe treatment of atrial fibrillation by heart ablation using a heat ablation device 54 comprises the steps of:
1) inserting a heat exchange assembly 11 as described herein, into the oesophagus of a subject,
2) adjusting the temperature of the heat exchange medium according to the power supplied to an ablation probe 55 of said ablation device and/or the temperature registered by an ablation probe 55), so as to lower the temperature of the oesophagus during heart ablation.

According to one aspect of the invention, the temperature of the oesophagus is maintained at a time-averaged temperature as defined above. According to one aspect of the invention, the time-averaged temperature of the oesophagus is less than 37 deg C., and preferably between 25 and 34 deg C. According to one aspect of the method, the temperature of the heat exchange medium is further adjusted according to the reading of a temperature sensor located in or on the balloon 4. According to another aspect of the method, the temperature of the heat exchange medium is further adjusted according to the desire of the physician.

### EXAMPLE

The present invention is illustrated by means of the following non-limiting example.

Twelve pigs were divided in three groups of four.

Group 1: No cooling device was used during RF ablation of the posterior wall of the left atrium.

Group 2: The device was introduced but instead of a cooling substance a substance at the same temperature of the pig's body temperature was used during RF ablation of the posterior wall of the left atrium. This group was useful to assess the safety of the device.

Group 3: The oesophageal cooling system device was used and cooling substance was administered during RF ablation of the posterior wall of the left atrium. In this group of pigs care was taken that the oesophagus was effectively being cooled.

During the study RF applications were powerful enough to guarantee oesophageal temperature increase without use of the device. After the procedure each pig was sacrificed and pathology samples of the anterior wall of the oesophagus were analysed to search for macroscopic or microscopic lesions showing harm to the oesophageal wall.

With this study we were able to prove that:
All pigs in groups 1 and 2 had at least one lesion in the anterior wall of the oesophagus while none of the pigs in group 3 (using the cooling system) had a lesion in the oesophagus.
The oesophageal cooling system is safe since none of the pigs in group 2 or 3 had any lesions or complications related to the system.
And that ablation with the system in place is not more harmful than without the system since the degree of lesions in the anterior wall of the oesophagus were the same in pigs of group 1 and group 2.

1. A heat exchange balloon assembly for cooling the esophagus during heat ablation treatment to the heart comprising:
an inflatable balloon adapted for insertion into the esophagus, provided with an exterior heat-transfer surface; and
a lumen within said inflatable balloon adapted to carry thermal exchange medium;
configured such that said heat-transfer surface conducts thermal energy between esophagus and said lumen,
wherein said balloon assembly is connected to a temperature controller provided with a means to receive data from an ablation device,
wherein said data comprises an indication of power supplied to an ablation probe of the ablation device, and
wherein said temperature controller and means to receive data are configured to adjust the temperature and/or flow rate of the heat exchange medium according to the power supplied to the ablation probe.

2. A heat exchange balloon assembly according to claim 1, wherein said data further comprises an indication of the temperature of the ablation probe, and the said temperature controller and means to receive data are configured to further adjust the temperature and/or flow rate of the heat exchange medium according to the temperature of the ablation probe.

3. A balloon assembly according to claim 1, wherein the data receiving means comprises a processor configured to process said data, and output a signal to the temperature controller useful for the adjustment of the temperature of the heat exchange medium.

4. A balloon assembly according to claim 1, wherein the temperature of heat exchange medium is adjusted to maintain a time-averaged temperature of the esophagus during ablation of less than 37 deg C.

5. A balloon assembly according to claim 4, wherein the time-averaged temperature of the esophagus during ablation is between 25 to 34 deg C.

6. A balloon assembly according to claim 1, comprising:
(a) a first elongate tubular body having a proximal end and a distal end;
(b) a second elongate tubular body having a proximal end and a distal end;
wherein said inflatable balloons is in fluid communication with the distal ends of the first elongate tubular body and the second elongate tubular body.

7. A balloon assembly according to claim 1, comprising:
(a) an elongate tubular body having a proximal end and a distal end;
(b) a fluid exit port;
wherein said inflatable balloon in fluid communication with the distal end of the elongate tubular body and said fluid exit port is in fluid communication with a distal end of the balloon.

8. A balloon assembly according to claim 7 wherein the fluid exit port is provided with a means to prevent thermal exchange medium draining into the esophagus until after the balloon has inflated.

9. A balloon assembly according to claim 1 wherein the balloon comprises an outer lumen configured to carry thermal exchange medium, and a hollow inner lumen.

10. A balloon assembly according to claim 1, wherein at least one elongate tubular body terminates in a tubing coupling.

11. A temperature controller suitable for use with a heat exchange balloon assembly according to claim 1, comprising means to adjust the temperature and/or flow rate of the heat exchange medium and a means to receive data from the ablation device.

12. A temperature controller according to claim 11 configured to adjust the temperature of the heat exchange medium according to the power output an ablation device and/or the temperature of the ablation probe.

13. A temperature controller according to claim 12, wherein the temperature of heat exchange medium is adjusted to maintain a time-averaged temperature of the esophagus during ablation of less than 37 deg C.

14. A temperature controller according to claim 13 wherein the time-averaged temperature of the esophagus during ablation is between 25 to 34 deg C.

15. An ablation system comprising a heat exchange balloon assembly according to claim 1, a means to receive data and an ablation device.

16. An ablation system according to claim 15 configured to adjust the temperature and/or flow rate of the heat exchange medium according to the power output of the ablation probe and/or the temperature of the ablation probe.

17. An ablation system according to claim 16, wherein the temperature of heat exchange medium is adjusted to maintain a time-averaged temperature of the esophagus during ablation of less than 37 deg C.

18. An ablation system according to claim 17 wherein the time-averaged temperature of the esophagus during ablation is between 25 to 34 deg C.

19. (canceled)

20. A method for the safe treatment of atrial fibrillation by heart ablation using a heat ablation device comprising the steps of:
a) inserting the balloon part of a heat exchange balloon assembly according to claim 1, into the esophagus of a subject,
b) adjusting the temperature and/or flow rate of the heat exchange medium according to the power supplied to an ablation probe of said ablation device, so as to lower the temperature of the esophagus during heat ablation treatment to the heart.

21. A method according to claim 20, further comprising the step of adjusting the temperature and/or flow rate of the heat exchange medium according to the temperature of the ablation probe 55), so as to lower the temperature of the esophagus during heat ablation treatment to the heart.

22. A method according to claim 20, wherein the temperature of the esophagus is maintained at a time-averaged temperature of less than or equal to 37 deg C.

23. A method according to claim 20, where the time-averaged temperature of the esophagus is between 25 and 34 deg C.

24. A method according to claim 20, wherein the temperature of the heat exchange medium is further adjusted according to the reading of a temperature sensor located in or on the balloon.

25. A method according to claim 20 wherein the temperature of the heat exchange medium is further adjusted according to the desire of the physician.
